(19) United States
(12) Patent Application Publication
(10) Pub. No.: US 2004/0022808 A1
(43) Pub. Date: Feb. 5, 2004

(54) Vaccine

(76) Inventors: Brigitte Desire Alberte Colau, Rixensart (BE); Marguerite Deschamps, Rixensart (DE)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/381,354

(22) PCT Filed: Oct. 1, 2001
(86) PCT No.: PCT/EP01/11326

(30) Foreign Application Priority Data
Oct. 2, 2000 (GB) 00240895

(51) Int. Cl. A61K 39/12

(52) U.S. Cl. 424/204.1

(57) ABSTRACT

In particular the present invention relates to vaccine formulations comprising split enveloped virus preparations, not split influenza virus preparations, in the manufacture of a vaccine formulation for intranasal delivery, methods of manufacture of such formulations and use of such vaccines in the prophylaxis or therapy of disease.

The present invention relates to novel vaccine formulations, methods of manufacture of such vaccines and the use of such vaccines in the prophylaxis or therapy of disease. In particular the present invention relates to vaccines comprising split enveloped virus preparations.

An enveloped virus is one in which the virus core is surrounded by a lipid-rich outer coat containing viral proteins.

In a particular embodiment the split enveloped virus of the vaccine formulation of the present invention is derived from Respiratory Syncitial Virus. The dangers of infection by enveloped viruses are illustrated by reference to RSV.

Human respiratory syncytial virus (RSV) is a member of the Paramyxoviridiae family of viruses and causes lower respiratory tract illness, particularly in young children and babies. Recent reports suggest that RSV is also an important pathogen in adults, particularly the elderly.

RSV is an enveloped virus with a non-segmented, negative strand ribonucleic acid (RNA) genome of 15,222 nucleotides that codes for 11 messenger RNAs, each coding for a single polypeptide. Three of the eleven proteins are transmembrane surface proteins: the G (attachment), F (fusion) and SH proteins. One protein is the virion matrix protein (M), three proteins are components of the nucleocapsid (N, P and L), and 2 proteins are nonstructural (NS1 and NS2). There are two further proteins M2-1 and M2-2. Two antigenically distinct sub-groups of RSV exist, designated subgroups A and B. Characterisation of strains from these sub-groups has determined that the major differences reside on the G proteins, while the F proteins are conserved.

Respiratory syncytial virus (RSV) occurs in seasonal outbreaks, peaking during the winter in temperate climates and during the rainy season in warmer climates.

RSV is a major cause of serious lower respiratory tract disease in children. It is estimated that 40-50% of children hospitalised with bronchiolitis and 25% of children hospitalised with pneumonia are hospitalised as a direct result of RSV infections. Primary RSV infection usually occurs in children younger than one year of age; 95% of children have serologic evidence of past infection by two years of age and 100% of the population do so by adulthood.

In infants and young children, infection progresses from the upper to the lower respiratory tract in approximately 40% of cases and the clinical presentation is that of bronchiolitis or pneumonia. Children two to six months of age are at greatest risk of developing serious manifestations of infection with RSV (primarily respiratory failure); however, children of any age with underlying cardiac or pulmonary disease, premature infants, and infants who are immunocompromised, are at risk for serious complications as well.

Symptomatic reinfection occurs throughout life and it has become increasingly apparent that RSV is an important adult pathogen as well, especially for the elderly.

RSV infection is almost certainly underdiagnosed in adults, in part because it is considered to be an infection of children. Consequently, evidence of the virus in adults is not sought in order to explain respiratory illness. In addition, RSV is difficult to identify in nasal secretions from individuals who have some degree of partial immunity to the virus, as do the large majority of adults. Young to middle-age adults typically develop a persistent cold-like syndrome when infected with RSV. Elderly individuals may develop a prolonged respiratory syndrome which is virtually indistinguishable from influenza, with upper respiratory symptoms which may be accompanied by lower respiratory tract involvement, including pneumonia. Institutionalised elderly populations are of particular concern, because they comprise large numbers of susceptible individuals clustered together. The spread of infection through such a population, many of whom have multiple medical problems which may predispose them to a more severe course of the disease, is difficult to control.

Furthermore, reports of recent studies evaluating the impact of RSV infection as a cause of hospitalisation in adults and in community dwelling healthy elderly further point to an important role of RSV infection in severe lower respiratory tract disease in these populations. RSV has been identified as one of the four most common pathogens causing severe lower respiratory tract disease resulting in hospitalisation of adults. It was also demonstrated that serious RSV infections in elderly persons are not limited to nursing homes or outbreak situations. Rather, RSV infection is a predictable cause of serious illness among elderly patients residing in the community. Similar to hospitalisations for influenza A, those related to RSV infections were associated with substantial morbidity, as evidenced by prolonged hospital stays, high intensive care admission rates, and high ventilatory support rates.

These studies point to the medical and economic need for an effective vaccine which can prevent severe complications of RSV infection, particularly in infants, adults and both community dwelling healthy and institutionalised elderly.

Similar dangers are posed by other enveloped viruses and there is still, therefore, a need for effective protection against infection and disease caused by such viruses.

The present invention provides the use of a split enveloped virus preparation which is not a split influenza virus preparation in the manufacture of a vaccine formulated for intranasal delivery.

Preferably the preparation comprises a pharmaceutically acceptable excipient.

The vaccine formulations of the present invention will be derived from enveloped viruses that are capable of being split. The enveloped virus may be derived from a wide variety of sources including viruses from human or animal origin. Where the virus is of non-human origin, such as a bovine origin, the virus is preferably a recombinant virus.

Preferably the vaccine formulations of the present invention are capable of stimulating a protective immune response against the enveloped virus after delivery.

Within the terms of this invention, the virus includes all enveloped viruses (excluding any influenza virus) illustrated by but not limited to:

1) Paramyxoviruses such as respiratory syncytial virus (A and B), parainfluenza virus (such as PIV-3), metapneumovirus, measles virus, mumps virus;

2) herpes viruses such as Epstein Barr virus, herpes simplex virus, cytomegalovirus;

3) flaviviruses such as dengue virus, yellow fever virus, tick-borne encephalitis virus, Japanese encephalitis virus;

4) togaviruses such as rubella virus, eastern, western, and Venezuelan equine encephalitis viruses; and

5) retroviruses such as human immunodeficiency virus.

The vaccine formulation of the invention optionally comprises more than one split virus preparation.

The vaccine formulation of the invention optionally comprises an antigen or antigens from pathogens in combination with the split preparation, to provide additional protection against disease. Suitable antigens, which do not need to come from split preparations, include for example antigens from any of the viruses listed above and pathogens which cause respiratory disease such as Streptococcus Pneumoniae.

The splitting of the virus is carried out by disrupting or fragmenting whole virus, infectious (wild-type or attenuated) or non-infectious (for example inactivated), with a disrupting concentration of a splitting agent which is generally, but not necessarily, a surfactant The virus to be split may also be a chimaeric recombinant virus, having immunogenic elements from more than one different virus.The disruption results in a full or partial solubilisation of all the virus proteins which alters the virus integrity.

Suitably a split virus is obtainable by contacting the virus with a splitting agent according to the present invention to fully disrupt the viral envelope. Other viral proteins become preferably fully or partially solubilised. The loss of integrity after splitting renders the virus non-infectious which can be assessed by suitable in vitro titration assays. Once disrupted the viral envelope proteins are generally no longer associated with whole intact virions. Other viral proteins are preferably fully or partially solubilized and are therefore not associated, or only in part associated, with whole intact virions after splitting.

The effect of the splitting agent on the viral envelope and virus proteins can be followed by the migration of the split virus and viral proteins in sucrose cushion experiments with visualization by Western Blot analysis and electron microscopy, as described herein.

The preparation of split vaccines according to the invention may involve the further steps of removal of the splitting agents and some or most of the viral lipid material. The process for the preparation of the split enveloped virus may further include a number of different filtration and/or other separation steps such as ultracentrifugation, ultrafiltration, zonal centrifugation and chromotographic steps in a variety of combinations, and optionally an inactivation step e.g. with formaldehyde or β-propiolactone or UV treatment which may be carried out before or after splitting. The splitting process may be carried out as a batch, continuous or semi-continuous process.

The split vaccines according to the invention generally contain membrane fragments and membrane envelope proteins as well as non-membrane proteins such as viral matrix protein and nucleoprotein in the absence of significant whole virions. Split vaccines according to the invention will usually contain most or all of the virus structural proteins although not necessarily in the same proportions as they occur in the whole virus. Preferred split virus preparations comprise at least half of the viral structural proteins, preferably all of such proteins. Subunit vaccines on the other hand consist essentially of one or a few highly purified viral proteins. For example a subunit vaccine could contain purified viral surface proteins which are known to be responsible for eliciting the desired virus neutralising antibodies upon vaccination.

In this invention various splitting agents such as non-ionic and ionic surfactants as well as various other reagents may be used. Examples of splitting agents useful in the context of the invention include:

1. Bile acids and derivatives thereof. Bile acids include cholic acid, deoxycolic acid, chenodeoxy colic acid, lithocholic acid ursodeoxycholic acid, hyodeoxycholic acid and derivatives like glyco-, tauro-, amidopropyl-1-propanesulfonic-, amidopropyl-2-hydroxy-1-propanesulfonic derivatives of forementioned bile acids, or N,N-bis(3DGluconoamidopropyl) deoxycholamide. A particular example is sodium deoxycholate—NADOC.

2. Non-ionic surfactants such as octoxynols (the Triton™ series), polyoxyethylene ethers such as polyoxyethylene sorbitan monooleate (Tween 80™), and polyoxythylene ethers or esters of general formula (I):

HO(CH₂CH₂O)ₙ—A—R (I)

wherein n is 1-50, A is a bond or —C(O)—, R is C₁₋₅₀ alkyl or phenyl C₁₋₅₀ alkyl, and combinations of two or more of these. Particular examples are; Tween80™:, Triton X-100™ and laureth 9;

3. Alkylglycosides or alkylthioglycosides, where the alkyl chain is between C6-C18 typical between C8 and C14, sugar moiety is any pentose or hexose or combinations thereof with different linkages, like 1−>6, 1−>5, 1−>4, 1−>3, 1−2. The alkyl chain can be saturated unsaturated and/or branched;

4. Derivatives of 3 above, where one or more hydroxylgroups, preferrably the 6 hydroxyl group is/are modified, like esters, ethoxylates, sulfates, ethers, carbonates, sulfosuccinates, Isethionates, ethercarboxylates, quarternary ammonium compounds;

5. Acyl sugars, where the acyl chain is between C6 and C18, typical between C8 and C12, sugar moiety is any pentose or hexose or combinations thereof with different linkages, like 1−>6, 1−>5, 1−>4, 1−>3, 1−2. The acyl chain can be saturated unsaturated and/or branched;

6. Sulphobetaines of the structure R-N,N-(R1,R2)3-amino-1-propanesulfonate, where R is any alkyl chain or arylalkyl chain between C6 and C18, typical between C8 and C16. The alkyl chain R can be saturated, unsaturated and/or branched. R1 and R2 alkyl chains between C1 and C4, typically C1;

7. Betains of the structure R-N,N-(R1,R2)-glycine, where R is any alkylchain between C6 and C18, typical between C8 and C16. The alkyl chain can be saturated unsaturated and/or branched. R1 and R2 are alkyl chains between C1 and C4, typically C1;

8. Polyoxyethylenealkylether of the structure R-(—O—CH2-CH2-)n-OH, where R is any alkylchain between C6 and C20 typical between C8 and C14.The alkyl chain can be saturated, unsaturated and/or branched. n is between 5 and 30 typical between 8 and 25;

9. N,N-dialkyl-Glucamides, of the Structure R-(N-R1)-glucamide, where R is any alkylchain between C6 and C18, typical between C8 and C12. The alkyl chain can be saturated unsaturated and/or branched or cyclic. R1 and R2 are alkyl chains between C1 and C6, typical C1. The sugar moiety might be modified with pentoses or hexoses;

10. Hecameg: (6-0-(N-heptyl-carbamoyl)-methyl-alpha-D-glucopyranoside);

11. Alkylphenoxypolyethoxyethanol of the structure R-C6H4-O—(—CH2-CH2-)n-OH, where R is any alkylchain between C6 and C18, typical C8. The alkyl chain can be saturated unsaturated and/or branched (n>=3);

12. Quaternary ammonium compounds of the structure R, —N⁺ (—R1, —R2, —R3), where R is any alkylchain between C6 and C20, typical C20. The alkyl chain can be saturated unsaturated and/or branched. R1, R2 and R3 are alkyl chains between C1 and C4, typical C1;

13. Sarcosyl: N-Laurylsarcosine Na salt;

14. CTAB (cetyl trimethyl ammonium bromide) or Cetavlon.

Most preferred are Naloc and Sarcosyl. Splitting agents are suitably incubated at room temperature with the virus to be split, for example overnight, to effect splitting. Combinations of splitting agents may be used, as appropriate.

The split vaccine preparation preferably contains at least one surfactant which may be in particular a non-ionic surfactant. The one or more non-ionic surfactants may be residual from the splitting process, and/or added to the virus after splitting. It is believed that the split antigen material is stabilised in the presence of a non-ionic surfactant, though it will be understood that the invention does not depend upon this necessarily being the case. Suitable stabilising non-ionic surfactants include the octoxynols (the Triton™ series), polyoxyethylene ethers such as polyoxyethylene sorbitan monooleate (Tween 80™), and polyoxythylene ethers or esters of general formula (I):

HO(CH₂CH₂O)ₙ—A—R (I)

wherein n is 1-50, A is a bond or —C(O)—, R is C₁₋₅₀ alkyl or phenyl C₁₋₅₀ alkyl, and combinations of two or more of these.

Preferred non-ionic surfactants from the Triton series include Triton X-100 (t-octylphenoxypolyethoxyethanol), Triton X-165, Triton X-205, Triton X-305 or Triton X-405 Triton N-101. Triton X-100 is particularly preferred.

Preferred non-ionic surfactants further include but are not restricted to polyoxyethylene ethers of general formula (I) above in particular: polyoxyethylene-9-lauryl ether, polyoxyethylene-9-stearyl ether, polyoxyethylene-8-stearyl ether, polyoxyethylene-4-lauryl ether, polyoxyethylene-35-lauryl ether, and polyoxyethylene-23-lauryl ether. Most preferably, the polyoxyethylene ether is polyoxyethylene-9-lauryl ether (laureth 9). Alternative terms or names for polyoxyethylene lauryl ether are disclosed in the CAS registry. The CAS registry number of polyoxyethylene-9 lauryl ether is: 9002-92-0. Polyoxyethylene ethers such as polyoxyethylene lauryl ether are described in the Merck index (12^{th} ed: entry 7717, Merck & Co. Inc., Whitehouse Station, N.J., USA; ISBN 0911910-12-3). Laureth 9 is formed by reacting ethylene oxide with dodecyl alcohol, and has an average of nine ethylene oxide units.

Preferably, the final concentration of stabilizing surfactant present in the final vaccine formulation is between 0.001 to 20%, more preferably 0.01 to 10%, and most preferably up to about 2% (w/v). Where one or more surfactants are present, these are generally present in the final formulation at a concentration of up to about 2% each, generally up to a concentration of about 1% each, typically at a concentration of up to about 0.6% each., and more typically in traces up to about 0.2% or 0.1% each. Any mixture of surfactants may be present in the vaccine formulations according to the invention.

The enveloped virus may be produced by replication on a suitable cell substrate, in serum or in a serum free process. Tissue culture-grown virus may be produced for example on human cells such as MRC-5, WI-38, HEp-2 or simian cells such as AGMK, Vero, LL_{C}-Mk₂, LLc-Mk2, FRhL, FRhL-2 or bovine cells such as MDBK, or canine cells such as MDCK, or primary cells such as chicken embryo fibroblasts, or any other cell type suitable for the production of a virus for vaccine purposes including clones derived from the above-mentioned cell lines.

The split vaccine preparation is suitably combined with a pharmaceutically acceptable excipient. The pharmaceutically acceptable excipients used may be those that are conventional in the field of vaccine preparation. The excipients used in any given vaccine formulation will be compatible both with each other and with the essential ingredients of the composition such that there is no interaction which would impair the performance of the ingredients and active agents, if any. All excipients must of course be non-toxic and of sufficient purity to render them suitable for human use. Suitable examples of excipients are well known in the art.

The vaccine formulation may preferably also include an adjuvant which may be a carrier and/or an immunostimulant. The adjuvant may be residual from the splitting process, and/or added to the virus after splitting. Suitable adjuvants for use in the vaccines of the present invention are well known in the art.

Thus a further aspect of the present invention provides the use of a split enveloped virus vaccine preparation which is not a split influenza virus preparation in combination with an adjuvant in the manufacture of a vaccine formulation for intranasal delivery. Preferably the preparation comprises a pharmaceutically acceptable excipient

The vaccine preparations of the present invention may be used to protect or treat a mammal susceptible to, or suffering from disease, by means of administering said vaccine via a nasal route. The invention extends to such methods of treatment and protection.

Apart from bypassing the requirement for painful injections and the associated negative effect on patient compliance because of “needle fear”, mucosal vaccination such as by an intransal method is attractive since it has been shown in animals that mucosal administration of antigens has a good efficiency of inducing protective responses at mucosal surfaces, which is the route of entry of many pathogens. In addition, it has been suggested that mucosal vaccination, such as intranasal vaccination, may induce mucosal immunity not only in the nasal mucosa, but also in distant mucosal sites such as the genital mucosa Despite much research in the field, safe and effective vaccines for intranasal delivery, which are suitable for use in humans, remain to be identified.

Intranasal administration according to the invention may be in a droplet, spray, or dry powdered form. Nebulised or aerosolised vaccine formulations also form part of this invention.

Any suitable adjuvant may be used in the present invention and in any suitable form, such as a solution, a non-vesicular solution, a suspension or a powder. Preferred adjuvants include those exemplified in WO99/52549 the whole contents of which are incorporated by reference. Preferred adjuvants include but are not limited to; Tween80™:, Triton X-100™, laureth 9 and combinations thereof.

The non-ionic surfactants may advantageously be combined with an immunostimulant such as a non-toxic derivative of lipid A including those described in U.S. Pat. No. 4,912,094, and GB 2,220,211 including non-toxic derivatives of monophosphoryl and diphosphoryl Lipid A such as 3-de-O-acylated monophosphoryl lipid A (3D-MPL) and 3-de-O-acylated diphosphoryl lipid A. A preferred combination is Laureth-9 combined with 3D-MPL. The above immunostimulants may also be used in formulations without non-ionic surfactants, where appropriate.

A preferred form of 3D-MPL is in the form of an emulsion having a small particle size less than 0.2 μm in diameter, and its method of manufacture is disclosed in WO 94/21292. Aqueous formulations comprising monophosphoryl lipid A and a surfactant have been described in WO9843670A2.

The bacterial lipopolysaccharide derived adjuvants to be formulated in the compositions of the present invention may be purified and processed from bacterial sources, or alternatively they may be synthetic. For example, purified monophosphoryl lipid A is described in Ribi et al 1986 (1986, Immunology and Immunopharmacology of bacterial endotoxins, Plenum Publ. Corp., NY, p407-419), and 3-O-Deacylated monophosphoryl or diphosphoryl lipid A derived from Salmonella sp. is described in GB 2220211 and U.S. Pat. No. 4,912,094. Other purified and synthetic lipopolysaccharides have been described (Hilgers et al., 1986, Int.ArchAllergy.Immnunol., 79(4):392-6; Hilgers et al., 1987, Immunology, 60(1):141-6; and EP 0 549 074 B1). A particularly preferred bacterial lipopolysaccharide adjuvant is 3D-MPL.

Accordingly, the LPS derivatives that may be used in the present invention are those immunostimulants that are similar in structure to that of LPS or MPL or 3D-MPL. In another aspect of the present invention the LPS derivatives may be an acylated monosaccharide, which is a sub-portion to the above structure of MPL.

In a further embodiment of the present invention the adjuvant is an ADP-ribosylating toxin or mutant thereof. Examples of such toxins are the Heat Labile Toxin (LT) from E. coli, and mutants thereof such as LTR192G, and fragments of these toxins such as the ganglioside-binding component (LTB).

Further preferred adjuvants include saponin adjuvants such as QS21.

An enhanced system involves the combination of a non-toxic lipid A derivative and a saponin derivative particularly the combination of QS21 and 3D-MPL as disclosed in WO 94/00153, or a less reactogenic composition where the QS21 is quenched with cholesterol as disclosed in WO 96/33739.

Preferred devices for intranasal administration of the vaccines according to the invention are spray devices. Suitable nasal spray devices are commercially available from Becton Dickinson, Pfeiffer GmBH and Valois.

Preferred spray devices for intranasal use do not depend for their performance on the pressure applied by the user. Pressure threshold devices are particularly useful since liquid is released from the nozzle only when a threshold pressure is attained. These devices make it easier to achieve a spray with a regular droplet size. Pressure threshold devices suitable for use with the present invention are known in the art and are described for example in WO 91/13281 and EP 311 863 B. Such devices are currently available from Pfeiffer GmbH and are also described in Bommer, R. Advances in Nasal drug delivery Technology, Pharmaceutical Technology Europe, September 1999, p26-33.

Preferred intranasal devices produce droplets (measured using water as the liquid) in the range 1 to 500 μm. Below 10 μm there is a risk of inhalation, therefore it is desirable to have no more than about 5% of droplets below 10 μm.

Bi-dose delivery is a further preferred feature of an intranasal delivery system for use with the vaccines according to the invention. Bi-dose devices contain two subdoses of a single vaccine dose, one sub-dose for administration to each nostril.

The invention also provides an intranasal delivery device comprising a split vaccine formulation of the present invention.

The invention provides in a further aspect a pharmaceutical kit comprising an intranasal administration device as described herein or comprising an intranasal administration device and a separate vaccine formulation for use with that device.

This aspect of the invention is not necessarily limited to spray delivery of liquid formulations. Vaccines according to the invention may be administered in other forms, for example, as a powder.

The vaccine formulations of the present invention may be used for both prophylactic and therapeutic purposes. Accordingly, the present invention provides for a method of treating a mammal susceptible to or suffering from an infectious disease. In a further aspect of the present invention there is provided a vaccine as herein described for use in medicine. Vaccine preparation is generally described in New Trends and Developments in Vaccines, edited by Voller et al., University Park Press, Baltimore, Md., U.S.A. 1978.

Vaccines may be delivered in any suitable dosing regime, such as a one dose or two dose regime. The vaccine may be used in naive and primed populations, i.e. in seronegative and seropositive individuals.

We prefer that the formulation comprises an adjuvant and/or is given to individuals already primed by exposure to virus.

The present invention further relates to a method of producing a vaccine formulation which comprises the steps of

(a) splitting an enveloped virus;

(b) optionally admixing the split enveloped virus preparation with a stabilising agent; and

(c) optionally admixing the split enveloped virus preparation with an adjuvant (wherein the adjuvant may also be a carrier and/or immunostimulant).

Suitably the method comprises steps (a) and (b), steps (a) and (c), or steps (a) (b) and (c). Suitably the stabilising agent comprising at least one surfactant selected from the group comprising polyoxyethylene sorbitan monooleate (ITWEEN80™); t-octylphenoxypolyethoxyethanol (TRITON X100™); polyoxyethylene-9-lauryl ether.

Optionally the vaccine produced in this way is admixed with a suitable carrier.

The invention also extends to methods for splitting enveloped viruses as described herein, comprising treatment of the virus with a suitable splitting agent.

The present invention is illustrated by, but not limited to, the following Figures and Examples, wherein:

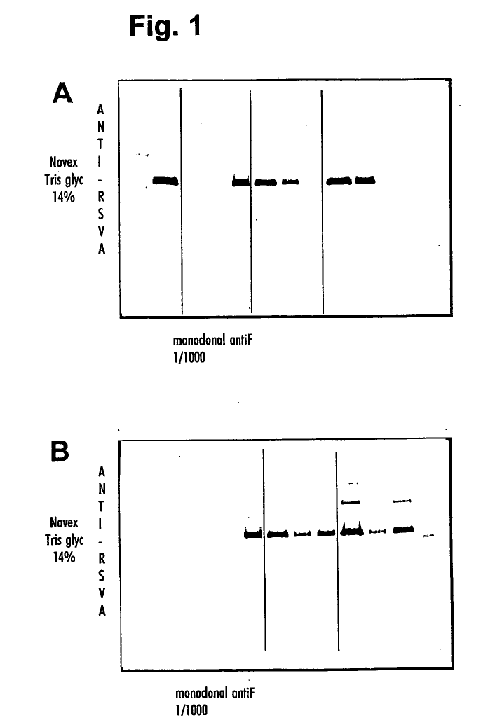

FIG. 1 illustrates a Western Blot of split RSVA with an anti F antibody;

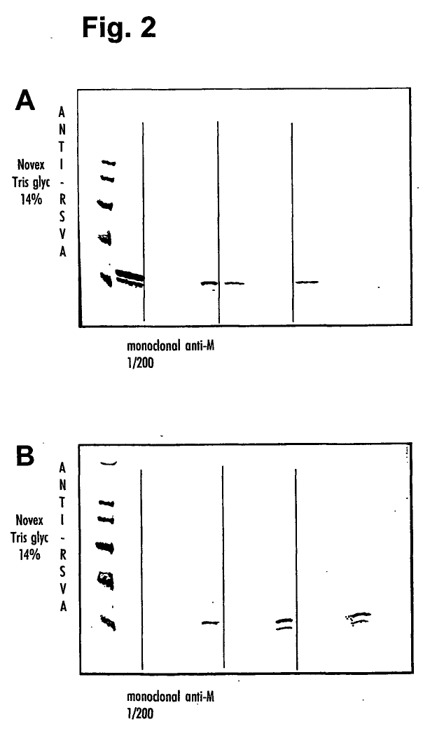

FIG. 2 illustrates a Western Blot of split RSVA with an anti-M2 antibody;

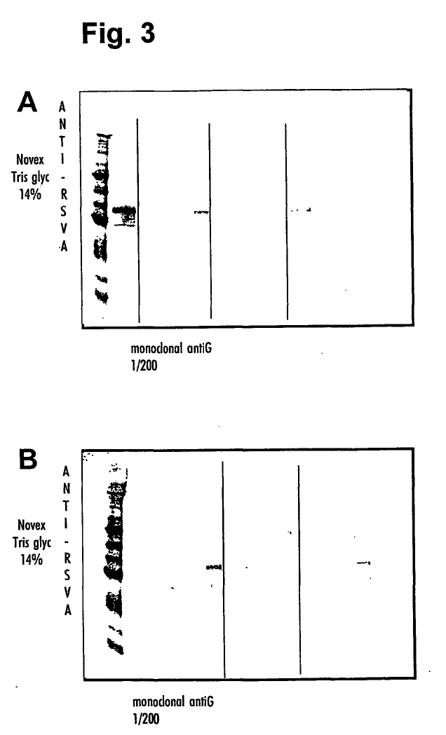

FIG. 3 illustrates a Western Blot of split RSVA with an anti G antibody;

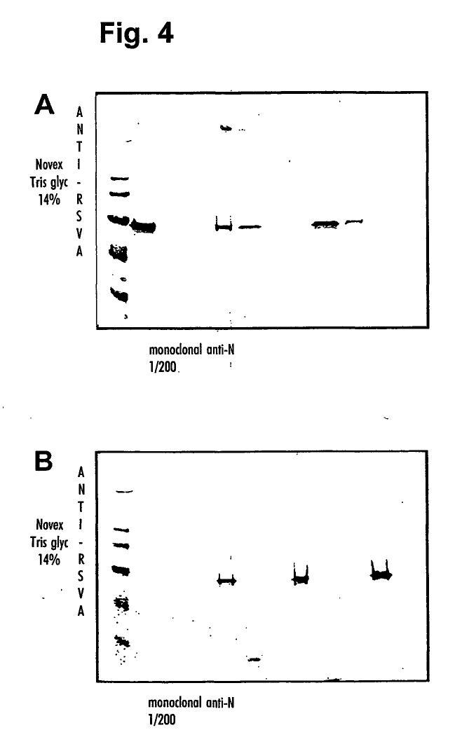

FIG. 4 illustrates a Western Blot of split RSVA with an anti N antibody;

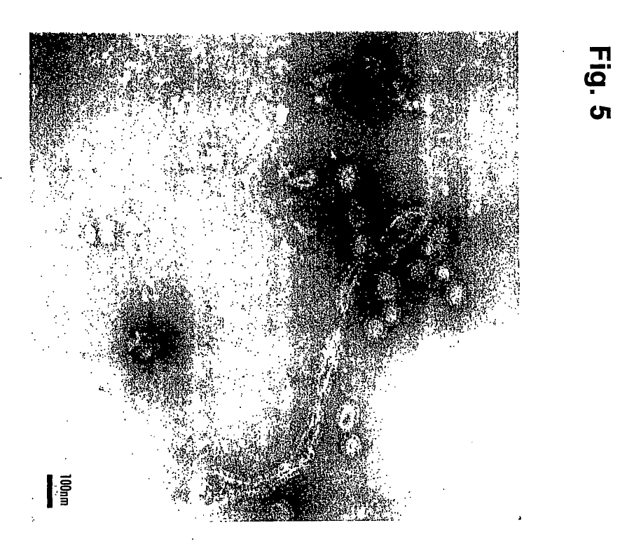

FIG. 5 illustrates RSV/A virus starting material visualised by EM;

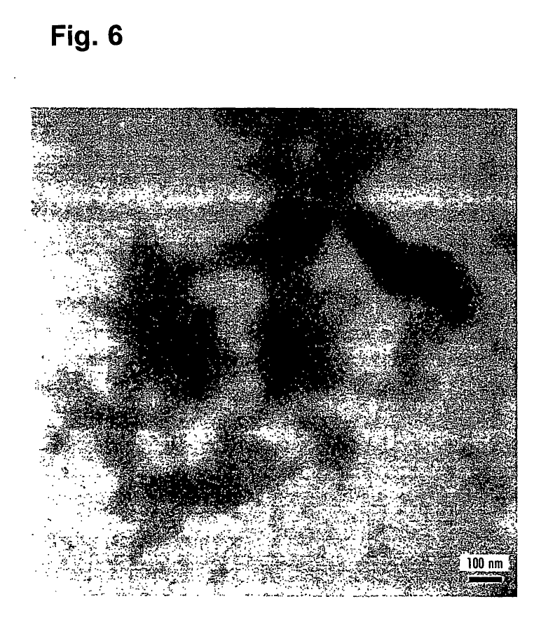

FIG. 6 illustrates RSV/A virus split with NaDOC visualised by EM;

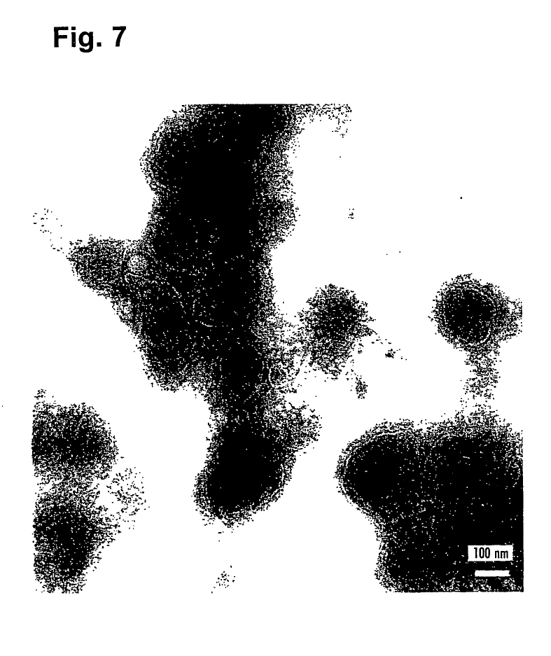

FIG. 7 illustrates PIV 3 virus starting material visualised by EM;

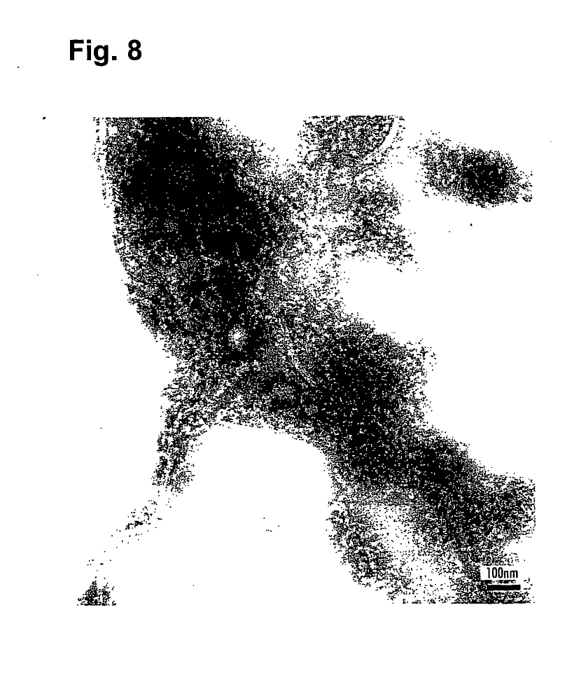

FIG. 8 illustrates PIV 3 virus split with NaDOC visualised by EM

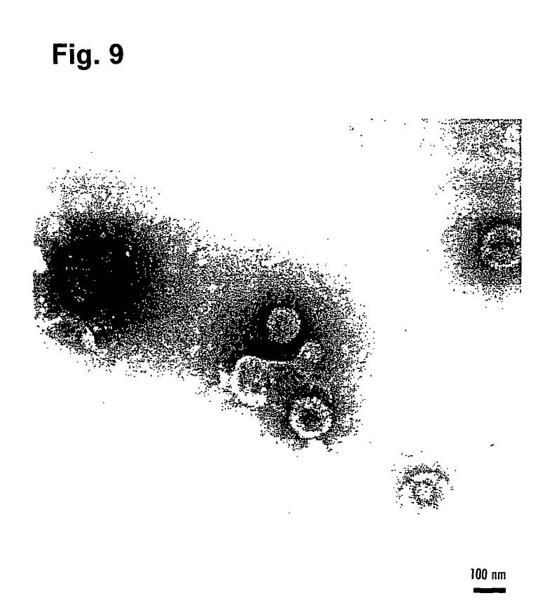

FIG. 9 illustrates HSV2 virus starting material visualised by EM

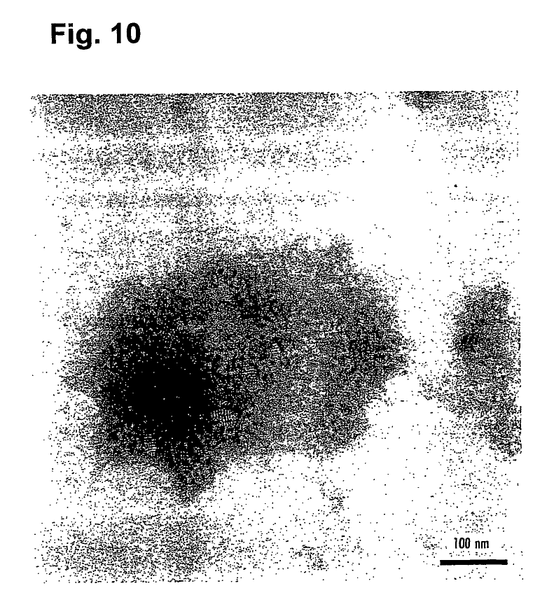

FIG. 10 illustrates HSV2 virus split with Sarcosyl visualized by EM

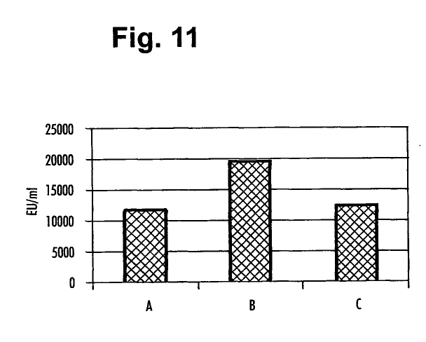

FIG. 11 illustrates Anti-FG Antibody (ELISA) Titers (Post II) in Primed Mice Immunized with Split RSV by the Intramuscular or Intranasal Routes;

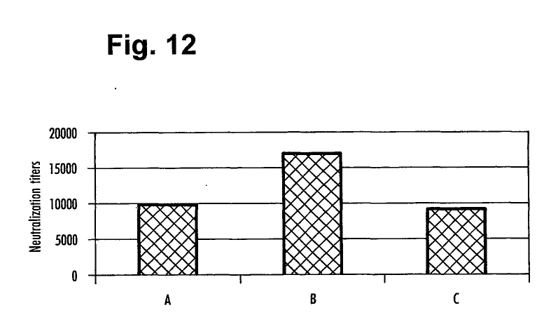

FIG. 12 illustrates Anti-RSV/A Neutralizing Antibody Titers (Post II) in Primed Mice Immunized with Split RSV by the Intramuscular or Intranasal Routes;

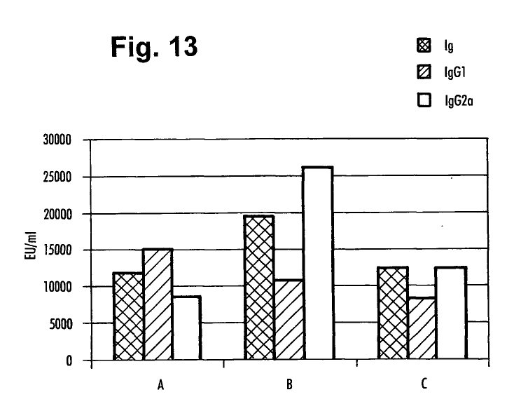

FIG. 13 illustrates Anti-FG IgG Isotype Responses (Post II) in Primed Mice Immunized with Split RSV by the Intramuscular or Intranasal Routes;

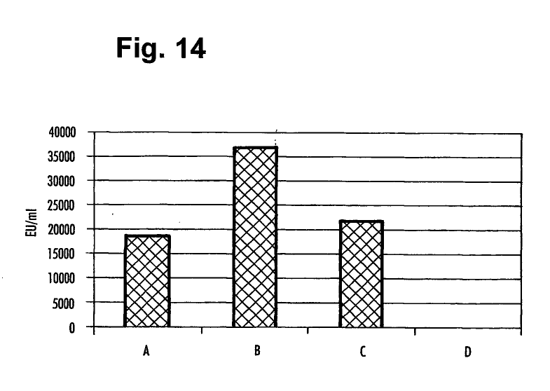

FIG. 14 illustrates Anti-FG Antibody (ELISA) Titers (Post I) in Primed Mice Immunized with Split RSV by the Intramuscular or Intranasal Routes;

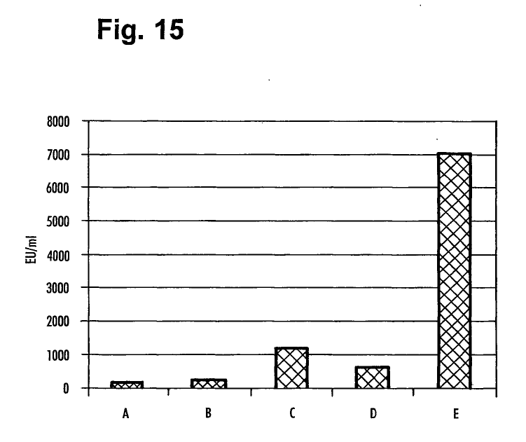

FIG. 15 illustrates Anti-FG Antibody (ELISA) Titers in Unprimed Mice Immunized with Split RSV by the Intranasal Route; and

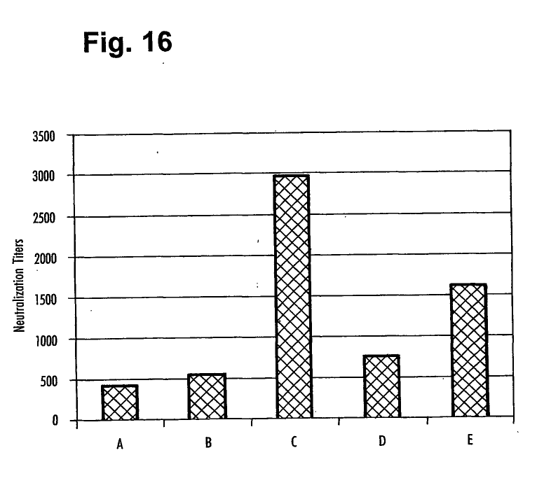

FIG. 16 illustrates Anti-RSV/A Neutralizing Antibody Titers in Unprimed Mice Immunized with Split RSV by the Intranasal Route.

### EXAMPLE 1

Generation of Split Viruses

Enveloped viruses derived from a variety of virus families are split by addition of splitting agents such as surfactants. The splitting is evaluated by characterization of the migration of the split viruses in sucrose gradients or cushions with visualization by SDS-PAGE analysis and by direct examination of split viral products using electron microscopic evaluation.

The split viruses described in this example include representatives of a variety of enveloped viral families. For example, members of the Paramyxoviridae family (respiratory syncytial viruses A and B, parainfluenza virus-3, mumps, and measles virus), Togaviridae family (rubella virus), and the Herpesviridae family (Epstein Barr virus, cytomegalovirus, or herpes simplex virus) are evaluated.

The act of disrupting the viral particle (splitting) is accomplished by addition of a splitting agent such as a surfactant at solubilizing concentrations to the cell-free viral preparations. In particular, bile acids and alkylglycosides are used as surfactants. The surfactants, alone or in various combinations, are added and incubated to allow the process to go to completion.

Evaluation of efficient splitting is conducted initially using sucrose gradient or cushion centrifugation. Briefly, surfactant-treated and non-treated samples are applied to sucrose gradients/cushions and the fractions analysed on SDS-PAGE gels. Migration of all types of virion proteins in the soluble fractions indicate efficient splitting. Samples deemed efficiently split by the sucrose-SDS-PAGE analysis are further analyzed by electron microscopy. The samples are visualized using standard negative staining techniques.

The following specific splitting experiments were carried out on RSV, HSV and PIV.

1.1 Cell Culture Conditions

Human wild-type RSV/A/Long and PIV-3 were replicated in VERO cells in a stationary serum free process. Before infection, VERO cells were grown for 4 days to confluency. Virus production conditions were adapted to each virus: MOI 0.03, 4 days for RSV/A, MOI 0.001, 3 days for HSV2 at 35° C. and MOI 0.01, 5 days for PIV-3 at 37° C. At the day of harvest, cell fluids were recovered after lysis and addition of stabiliser and were immediately stored at −70° C.

1.2 Virus Purification

After clarification by centrifugation at 1,000×g for 10 min, virus particles were pelleted from the supernatant by a PEG 6000 precipitation. The pellet was resuspended in Tris 50 mM—NaCl 50 mnM—MgSO4 2 mM pH 7.5 buffer followed by a benzonase treatment. This solution was ultrafiltrated on a 500 kD AGT membrane against 5 volumes of phosphate-buffered saline then diafiltred against 5 volumes of phosphate buffer pH 7.5.

Intact viral particles were produced as confirmed by EM and centrifugation on a sucrose cushion as described herein. The protein concentration was determined.

1.3 Virus Splitting

The viral particles were split by addition of a splitting agent to the cell-free viral preparation.

To be effective a detergent must be used above its critical micellar concentration, cmc. All detergents were used at a final concentration above their cmc value. The ratio D/P (detergent/protein ratio) was studied. The splitting was achieved successfully with a ratio D/P≧25, which is preferred.

The following detergents were used at a 2% concentration to split the virus particles; Sodium Deoxycholate, Sarkosyl, Plantacare and Laureth 9.

After splitting, the solutions were dialyzed against formulation buffer (PO4 10 mM/NaCl 150 mM pH7.5) for removal of excess detergent.

The splitting process is summarised below for RSV, by way of example.

RSV-A: Virus Purification Flow Sheet

Harvest Clarification
Centrifugation in Beckman JA10 rotor at 3500 RPM (1000 × g) + 4° C. for 10 min.
→ Clarified supernatant.
↓
10% PEG 6000 precipitation.
Slow stirring 1h30 at +4° C.
Centrifugation 3500 RPM (1000 × g) − 20 min
Pellet resuspended in Tris 50 mM-NaCl 50 mM-MgSO4 2 mM pH 7.5 buffer.
↓
Benzonase treatment.
At 125 unit/ml
Minimum 4 hours incubation under stirring
↓
ULTRAFILTRATION: AGT-VAGE4A-500 Kd-420 cm²
5 vol against PO4(Na) 10 mM-NaCl 150 mM pH 7.5
followed by 5 volumes of PO4 (Na) 20 mM pH 7.5
↓
Splitting
Detergent addition =>≦2% final-incubation O/N at room temperature under slow stirring
↓
Clarification
Dead-end filtration on Sartopure 300 (GF2-depth filter 1.2 μm)
↓
ULTRAFILTRATION: detergent elimination-concentration
5 vol PO4(Na) 20 mM pH 7.5
then 5 vol PO4(Na) 10 mM/NaCl 150 mM pH 7.5
↓
Split bulk

1.4 Split Virus Characterisation

Integrity of starting viruses and split quality was determined by ultracentrifugation on a 30% sucrose cushion (1 h at 50.000 rpm in TL100 Beckman rotor). Fractions were analyzed by specific Western blotting assays; electron microscopy and infectivity titer were performed on some of these fractions.

1.4.1 Ultracentrifugation:

After half filling a centrifuge tube with the 30% sucrose solution (450 μl), the sample (450 μl) to be analyzed was laid gently and carefully onto this sucrose cushion then run for 1 hour at 50.000 rpm at +4° C. in a Beckman TL100 rotor. After centrifugation, the tube was drained in 3 parts The upper phase (300 μl) is referred to as the ‘supernatant’. The middle phase (300 μl) is the interface phase between the sample and the sucrose cushion, called herein the ‘middle’. The lower phase (300 μl) is the bottom solution with the resuspended pellet when centrifugation has been performed on integer virus; called the ‘pellet’.

These 3 fractions were further analysed.

1.4.2 Western Blotting Analysis

This analysis allows the integrity of the virus to be checked (pellet fraction positive) and the efficacy of the split to be determined (suitably, supernatant fraction positive for all or most structural proteins such as the envelope proteins).

Specific antibodies were used for the characterization of specific viral proteins.

For RSV-A non-split and split fractions were analyzed for the anti F protein (surface protein); anti G protein (surface protein); anti N protein (nucleocapsid) and anti M protein (matrix) content.

For PIV-3 virus, the non-split and split fractions were analyzed for their HN protein content with a monoclonal antibody and the F, M, HN proteins content with a polyclonal antibody.

For HSV the non-split and split fractions were analyzed for their G protein, tegument protein and capsid protein with antibodies.

Criteria for Splitting

The presence of a positive Western Blot (WB) signal against all four proteins tested in the pellet fraction and absence of a signal in the two other fractions before splitting suggests the presence of whole intact virus in the viral preparation.

The split was considered effective when the envelope was disrupted, and envelope proteins were detected in the supernatant and/or middle fraction. For RSV, splitting was effective when F or G, for example, were detected in S or M fractions. Preferably F and G were located substantially in the S and/or M layers, and not in the pellet.

Summary of Results:

Results are shown in FIGS. 14 for RSVA.

In all Western Blot results, ‘Split —O’ means the virus before splitting. ‘S’, ‘M’ and ‘P’ refer to ‘Supernatant’, ‘Middle’ and ‘Pellet’ fractions taken after ultracentrifugation of the sample on a sucrose cushion respectively. Numbering of lanes is left to right. Volumes refer to the quantity of sample deposited on SDS-PAGE gels.

FIG. 1 illustrates a western blot of split RSVA probed with mAb B4 (anti-F).

| In the upper panel: | | | In the lower panel: | | |
|---|---|---|---|---|---|
| 1 | STD | 10 μl | 1 | STD | 10 μl |
| 2 | Split-O | 10 μl | 2 | Sample buffer | 10 μl |
| 3 | Split O-S | 10 μl | 3 | Split O-S | 10 μl |
| 4 | Split O-M | 10 μl | 4 | Split O-M | 10 μl |
| 5 | Split O-P | 10 μl | 5 | Split O-P | 10 μl |
| 6 | Split DOC-S | 10 μl | 6 | Split planta-S | 10 μl |
| 7 | Split DOC-M | 10 μl | 7 | Split planta-M | 10 μl |
| 8 | Split DOC-P | 10 μl | 8 | Split planta-P | 10 μl |
| 9 | Split sarco-S | 10 μl | 9 | Split laureth9-S | 10 μl |
| 10 | Split sarco-M | 10 μl | 10 | Split laureth9-M | 10 μl |
| 11 | Split sarco-P | 10 μl | 11 | Split laureth9-P | 10 μl |
| | | | 12 | STD | 10 μl |

FIG. 2 illustrates a western blot of split RSVA probed with an anti-M monoclonal;

| In the upper panel: | | | Lower panel: | | |
|---|---|---|---|---|---|
| 1 | STD | 10 μl | 1 | STD | 10 μl |
| 2 | Split-O | 20 μl | 2 | Sample buffer | 20 μl |
| 3 | Split O-S | 20 μl | 3 | Split O-S | 20 μl |
| 4 | Split O-M | 20 μl | 4 | Split O-M | 20 μl |
| 5 | Split O-P | 20 μl | 5 | Split O-P | 20 μl |
| 6 | Split DOC-S | 20 μl | 6 | Split planta-S | 20 μl |
| 7 | Split DOC-M | 20 μl | 7 | Split planta-M | 20 μl |
| 8 | Split DOC-P | 20 μl | 8 | Split planta-P | 20 μl |
| 9 | Split sarco-S | 20 μl | 9 | Split laureth9-S | 20 μl |
| 10 | Split sarco-M | 20 μl | 10 | Split laureth9-M | 20 μl |
| 11 | Split sarco-P | 20 μl | 11 | Split laureth9-P | 20 μl |

FIG. 3 illustrates a western blot of split RSVA probed with an anti-G monoclonal;

| Upper panel: | | | Lower panel: | | |
|---|---|---|---|---|---|
| 1 | STD | 10 μl | 1 | STD | 10 μl |
| 2 | Split-O | 20 μl | 2 | Sample buffer | 20 μl |
| 3 | Split O-S | 20 μl | 3 | Split O-S | 20 μl |
| 4 | Split O-M | 20 μl | 4 | Split O-M | 20 μl |
| 5 | Split O-P | 20 μl | 5 | Split O-P | 20 μl |
| 6 | Split DOC-S | 20 μl | 6 | Split planta-S | 20 μl |
| 7 | Split DOC-M | 20 μl | 7 | Split planta-M | 20 μl |
| 8 | Split DOC-P | 20 μl | 8 | Split planta-P | 20 μl |
| 9 | Split sarco-S | 20 μl | 9 | Split laureth9-S | 20 μl |
| 10 | Split sarco-M | 20 μl | 10 | Split laureth9-M | 20 μl |
| 11 | Split sarco-P | 20 μl | 11 | Split laureth9-P | 20 μl |

FIG. 4 illustrates a western blot of split RSVA probed with an anti-N monoclonal;

| Upper panel: | | | Lower panel: | | |
|---|---|---|---|---|---|
| 1 | STD | 10 μl | 1 | STD | 10 μl |
| 2 | Split-O | 20 μl | 2 | Sample buffer | 20 μl |
| 3 | Split O-S | 20 μl | 3 | Split O-S | 20 μl |
| 4 | Split O-M | 20 μl | 4 | Split O-M | 20 μl |
| 5 | Split O-P | 20 μl | 5 | Split O-P | 20 μl |
| 6 | Split DOC-S | 20 μl | 6 | Split planta-S | 20 μl |
| 7 | Split DOC-M | 20 μl | 7 | Split planta-M | 20 μl |
| 8 | Split DOC-P | 20 μl | 8 | Split planta-P | 20 μl |
| 9 | Spllt sarco-S | 20 μl | 9 | Split laureth9-S | 20 μl |
| 10 | Split sarco-M | 20 μl | 10 | Split laureth9-M | 20 μl |
| 11 | Split sarco-P | 20 μl | 11 | Split laureth9-P | 20 μl |

The presence of a signal in the medium and supernatant fractions and hardly any band in the pellet fraction after splitting for the F and G proteins shows that the vial envelope was completely disrupted. The presence of a signal in all fractions for the N and M proteins shows the presence of these proteins in the split preparations. These results suggest that all four detergents tested lead to RSVA split virus.

Analysis of the signals against all four proteins in all fractions and in particular the comparative signals against N and M proteins after splitting in the medium and supernatant fractions suggests that, in the conditions tested, NaDOC and Sarkosyl not only lead to split virus but are also able to disrupt all viral structures and solubilize structural and non-structural proteins.

Similar results were obtained with split PIV, HSV and measles which may be successfully split using 2% Nadoc or sarcosyl, for example.

NaDoc and Sarkosyl are preferred splitting agents for all viruses.

1.5 In vitro Viral Titrations

The loss of integrity after splitting renders the virus non-infectious. Analysis of the successful disruption of virus is shown by the loss of 10⁶ log or more in viral titer following splitting.

1.6 Electron Microscopy (EM) Analysis

Electron microscopy analysis was performed using a standard two-step negative staining method using Na phosphotungstate as contrasting agent (Hayat and Miller, 1990, Negative Staining, McGraw, ed. Hill). Grids were examined to assess the splitting pattern of the material.

Analysis by electron microscopy of non-split and NaDoc or Sarkosyl split RSVA, HSV2 and PIV3 virus preparations supports the observations made by Western Blot analysis. To illustrate results, the RSV, PIV and HSV data are shown.

FIG. 5 illustrates RSV/A starting material visualised by EM. FIG. 6 illustrates RSV/A after splitting with NaDOC. FIG. 7 illustrates PIV 3 starting material visualised by EM. FIG. 8 illustrates PIV 3 after splitting with NADOC. FIG. 9 illustrates HSV2 starting material visualised by EM. FIG. 10 illustrates HSV2 after splitting with Sarcosyl.

The non-split virus (whole intact virus) contained relatively well preserved or lightly damaged viral particles and some amorphous material. NaDoc or Sarkosyl split viruses showed the appearance of a heterogeneous spread of amorphous material, aggregated to various extent. Similar data were obtained with all viruses tested, RSV, PIV and HSV. In addition, few identifiable structures from viral envelope or nucleoproteic origin were observed with RSV or PIV.

### EXAMPLE 2

Immunogenicity of Split Vaccines in Mice.

Split RSV and/or PIV preparations are used as immunogens to vaccinate mice to assess the immunogenicity of these preparations. Briefly, 8 week old female mice are immunized with the intranasal split vaccine preparations. A non-adjuvanted control is included. Two doses are given at an interval of several weeks.

Two weeks following the final dose, the animals are sacrificed and blood, spleen cells, and/or nasal washes are collected. The virus-specific humoral immune response in serum is assessed by testing the mouse serum in virus-specific ELISA assays. In addition, the isotype profile of the antibody response is determined using Isotype-specific assays. The presence of neutralizing antibodies in the serum is assessed using a specific virus neutralization assay. Induction of a relevant local immune response may be assessed by assay of neutralizing antibodies in the nasal washes or alternatively assay of virus-specific IgA in the nasal washes.

Induction of virus specific cellular immune responses is assessed by in vitro stimulation of harvested spleen cells and measurement of cellular proliferation (tritiated thymidine uptake) and/or secretion of IL-5 and IFNγ by the stimulated cells.

The impact of the variables in the experiment is assessed with specific attention paid to the quality and magnitude of the response induced by the split formulations.

2.1 Split RSV Formulations

The following series of experiments exemplifies that split viruses such as RSV induce a potent immune response when administered by the intranasal IN route. In order to more accurately reflect the immune status of either a pediatric (naive) or elderly (primed) population, the immunogenicity was evaluated in either primed or unprimed animals and immunogenicity was demonstrated in both populations. IM delivery was used for comparison.

In the first set of experiments, 8 week old female Balb/c mice were used to test the immunogenicity of the split RSV preparation administered by either the IM or IN routes. Priming was accomplished by administration of 3×10⁵ plaque forming units (pfu) of live RSV virus administered intranasally in a volume of 60 μ(2×30 μl). Three weeks following priming, animals were vaccinated with RSV split antigen. Quantitation of the RSV split product was based on an RSV F protein specific ELISA which quantitates the F protein in the split product compared to a recombinant FG protein standard. Group A mice were immunized with 2 doses of RSV split antigen containing 4.2 μg F protein in 100 μl administered by the intramuscular route at a 21 day interval. Group B mice were immunized with 2 doses of RSV split antigen containing 4.2 μg F protein adjuvanted with 50 μg Al(OH)₃ administered in 100 μl by the intramuscular route at a 21 day interval. Group C mice were immunized with a first dose of RSV split antigen containing 2.7 μg F protein in 60 μl and a second dose administered 21 days later of RSV split antigen containing 4 μg F protein in 60 μl by the intranasal route. Two weeks following the last dose all animals were sacrificed and the immune response evaluated.

The results of the experiment are summarized in FIGS. 11-16. The first immune read outs used to evaluate the immune response were ELISA assays which measure the total RSV FG-specific immunoglobulin (Ig) or the FG-specific IgG isotypes (IgG₁ and IgG_{2A}) present in the sera of vaccinated animals. In these assays 96 well dishes are coated with recombinant RSV FG antigen and the animal sera are serially diluted and applied to the coated wells. Bound antibody is detected by addition of a biotinylated anti-mouse Ig, IgG₁, or IgG_{2A}, followed by an amplification with peroxidase-conjugated streptavidin. Bound antibody is revealed upon addition of OPDA substrate, followed by treatment with 2 N H₂SO₄ and measurement of the optical density (OD)at 490 nm. The antibody titer is calculated from a reference using SoftMax Pro software (using a four parameter equation) and expressed in EU/ml.

In addition to ELISA assays, neutralization assays were included to further characterize the quality of the immune response induced by the immunizations. For the neutralization assay, two-fold dilutions of animal sera were incubated with RSV/A virus (3000 pfu) and guinea pig complement for 1 hour at 37° C. in 96 well tissue culture dishes. Hep-2 cells (10⁴ cells/well) were added directly to each well and the plates incubated for 4 days at 37° C. The supernatants were aspirated and a commercially available WST-1 solution was added to each well. The plates were incubated for an additional 18-24 hours at 370° C. The OD was monitored at 450 nm and the titration analysed by linear regression analysis. The reported titer is the inverse of the serum dilution which resulted in 50% reduction of the maximal OD observed for uninfected cells.

FIG. 11 shows the results obtained using the total Ig ELISA read out. In primed mice a potent anti-FG antibody response was induced by 2 vaccinations with split RSV antigen administered IM (Groups A,B) or IN (Group C).

Specifically, FIG. 11 shows anti-FG antibody (ELISA) titers (post secondary vaccination) in mice primed with live RSV and immunized with split RSV by the intramuscular (IM) or intranasal (IN) routes. Group A received 2 doses of 4.2 μg each split RSV IM. Group B received 2 doses of 4.2 μg each split RSV adjuvanted with alum IM. Group C received 2 doses of 2.7 and 4.0 μg respectively split RSV IN.

FIG. 12 shows the results of the neutralization assay. A potent virus neutralizing antibody response was induced in these primed animals by either IM or IN vaccination with 2 doses of the split RSV product

Specifically, FIG. 12 shows Anti-RSV/A Neutralizing antibody titers (post secondary vaccination) in mice primed with live RSV and immunized with split RSV by the intramuscular (IM) or intranasal (IN) routes. Group A received 2 doses of 4.2 μg each split RSV IM. Group B received 2 doses of 4.2 μg each split RSV adjuvanted with alum IM. Group C received 2 doses of 2.7 and 4.0 μg respectively split RSV IN.

FIG. 13 shows the results of the isotype analysis. In animals primed intranasally the ratio of IgG₂ₐ:IgG₁ is increased compare to data generated in unprimed mice (see below), suggesting a tendency towards a more Th1-like response when mice are primed with live virus (i.e. natural situation in elderly populations).

Specifically, FIG. 13 shows Anti-FG IgG Isotype (ELISA) responses (post secondary vaccination) in mice primed with live RSV and immunized with split RSV by the intramuscular (IM) or intranasal (IN) routes. Group A received 2 doses of 4.2 μg each split RSV IM. Group B received 2 doses of 4.2 μg each split RSV adjuvanted with alum IM. Group C received 2 doses of 2.7 and 4.0 μg respectively split RSV IN.

FIG. 14 demonstrates that even after a single dose of antigen a strong immune response is generated in response to IN vaccination with split RSV in primed populations. Thus, in primed populations split RSV is a potent immunogen inducing high titer antibody responses following IN vaccination.

Specifically, FIG. 14 shows anti-FG antibody (ELISA) titers (post primary vaccination) in mice primed with live RSV and immunized with split RSV by the intramuscular (IM) or intranasal (IN) routes. Group A received 2 doses of 4.2 μg each split RSV IM. Group B received 2 doses of 4.2 μg each split RSV adjuvanted with alum IM. Group C received 2 doses of 2.7 and 4.0 μg respectively split RSV IN.

Group D was primed only and did not receive a vaccination—antibody titers reported for this group are below the detection level and measured at 21 days post-priming.

In the second series of experiments unprimed mice were used to document the effect of antigen dose and adjuvantation on the immunogenicity of the split RSV product. Mice received split RSV antigen containing 2.4 μg F protein (delivered in 60 μl-2×30 μl) for the first dose. For the second dose delivered 30 days later the mice received split RSV antigen containing 3.5 μg F protein. The IN split RSV were either administered without adjuvant or adjuvanted by addition of 5 μg E. coli labile toxin (LT) or with polyoxyethylene-9-lauryl ether 0.5%(herein ‘Laureth 9’). A control group was immunized intranasally with whole purified RSV virus containing 2.0 μg F protein in the first dose and 3.5 μg F protein in the second dose. Two weeks after the final vaccination the animals were sacrificed and the immune response evaluated.

As shown in FIGS. 15 and 16 antibody responses are induced by the IN formulations in unprimed mice. While the ELISA read out (FIG. 15) suggests that the responses to IN vaccination are lower than those induced by IM, the neutralization read out (FIG. 16) suggests that the LT adjuvanted split RSV IN formulation is at least as good as IM in inducing neutralizing antibodies and that the other formulations are also comparable to IM. Thus, in unprimed mice split RSV administered by the IN route is also immunogenic.

Specifically, FIG. 15 shows anti-FG antibody (ELISA) titers (post secondary vaccination) in unprimed mice immunized with split RSV by the intranasal (IN) or intramuscular (IM) routes. Group A received 2 doses of 2.4 and 3.5 μg each split RSV IN. Group B received 2 doses of 2.4 and 3.5 μg each split RSV adjuvanted with Laureth 9 IN. Group C received 2 doses of 2.4 and 3.5 μg each split RSV adjuvanted with LT IN. Group D received 2 doses of 2.0 and 3.5 μg each purified whole virus IN. Group E received 2 doses of 4.2 μg each split RSV IM.

FIG. 16 shows anti-RSV/A Neutralizing antibody titers (post secondary vaccination) in unprimed mice immunized with split RSV by the intranasal (IN) or intramuscular (IM) routes. Group A received 2 doses of 2.4 and 3.5 μg each split RSV IN. Group B received 2 doses of 2.4 and 3.5 μg each split RSV adjuvanted with Laureth 9 IN. Group C received 2 doses of 2.4 and 3.5 μg each split RSV adjuvanted with LT IN. Group D received 2 doses of 2.0 and 3.5 μg each purified whole virus IN. Group E received 2 doses of 4.2 μg each split RSV IM.

In summary, these experiments have demonstrated that split RSV antigen is strongly immunogenic in both naive and primed populations. In addition, these experiments have shown that split RSV can be administered effectively by the intranasal route, and is immunogenic.

1. The use of a split enveloped virus preparation which is not a split influenza virus preparation in the manufacture of a vaccine formulation for itranasal delivery.

2. The use according to claim 1 wherein the split enveloped virus preparation is either singly or a mixture of respiratory syncytial virus, parainfluenza virus, measles and herpes simplex virus.

3. The use according to claim 1 or claim 2 wherein the split enveloped virus preparation comprises viral membrane fragments, viral membrane envelope proteins, viral matrix and nucleoproteins.

4. The use according to any one of claims 1-3 which additionally comprises one or more residual splitting agents.

5. The use according to claim 4 wherein the residual splitting agent is selected from the group consisting of: laureth 9, NaDOC, Sarcosyl group, Tween 80™, and Triton 100™.

6. Use according to claim 5 wherein the splitting agent is NaDOC or Sarcosyl.

7 The use according to any one of claims 1 to 6 which additionally comprises a stabilising agent.

8 The use according to claim 7 wherein the stabilising agent is a surfactant.

9 The use according to claim 8 wherein the surfactant is either singly or a mixture of polyoxyethylene sorbitan monooleate (TWEEN80™), t-octylphenoxypolyethoxyethanol (TRITON X100™) and polyoxyethylene-9-lauryl ether.

10 The use as claimed in any one of the preceding claims which additionally comprises an adjuvant.

11 A method of producing an intranasal vaccine formulation which is not a split influenza virus preparation as claimed in any one of the preceding claims which comprises the steps of
(a) splitting an enveloped virus; and
(b) optionally admixing the split enveloped virus preparation with a stabilising agent; and
(c) optionally admixing the split enveloped virus preparation with an adjuvant (carrier and/or immunostimulant).

12 A method of producing a vaccine formulation as claimed in claim 11 wherein the stabilising agent is at least one surfactant selected from the group comprising polyoxyethylene sorbitan monooleate (TWEEN80™); t-octylphenoxypolyethoxyethanol (TRITON X100™); polyoxyethylene-9-lauryl ether.

13 Use of a split enveloped virus vaccine preparation, which is not a split influenza virus preparation in the manufacture of an intranasal vaccine formulation for the prophylaxis or treatment of disease.

14 A kit for delivery of an intranasal vaccine formulation as claimed in any one of claims 1-10 comprising:
(a) a split enveloped virus preparation; and
(b) an intranasal delivery device.

15 An intranasal delivery device comprising a vaccine according to any of claims 1-10.

16 A device according to claim 15 which is a pressure threshold device.

17 A method for protecting or treating a mammal susceptible to, or suffering from disease caused by an enveloped virus, the method comprising administering a vaccine according to claims 1-10 via a nasal route.

18 A method, use, kit or device according to any preceding claim, wherein the vaccine formulation is immunogenic in seropositive and seronegative individuals.
